(19) United States
(12) Patent Application Publication
(10) Pub. No.: US 2005/0238710 A1
(43) Pub. Date: Oct. 27, 2005

(54) Vitamin replacement as a hangover ameliorative

(76) Inventor: Philip Connolly, Lake Arrowhead, CA (US)

Correspondence Address: Paul M. Denk, Ste. 170, 763 S. New Ballas Road, St. Louis, MO, 63141, US

(21) Appl. No.: 11/110,122

(22) Filed: Apr. 20, 2005

Related U.S. Application Data
(60) Provisional application No. 60/565,023, filed on Apr. 23, 2004.

(51) Int. Cl. A61K 31/714; A61K 31/59; A61K 31/525; A61K 31/51

(52) U.S. Cl. 424/464; 514/52; 514/167; 514/251; 514/276; 514/350; 514/356

(57) ABSTRACT

Metabolizing alcohol requires B vitamins and other nutrients in the liver. A person who drinks a large quantity of alcohol depletes those vitamins and nutrients. With lowered vitamins and nutrients a person feels uncomfortable following drinking, commonly called a hangover. A hangover ameliorative has four pills of vitamins and nutrients taken as two pills before drinking and two pills before sleeping. The hangover ameliorative overloads a person with nutrients consumed during metabolization of alcohol resulting in better sleep and less chance for a headache.

### CROSS REFERENCE TO RELATED APPLICATION

This nonprovisional patent application claims priority to the provisional patent application having Ser. No. 60/565,023, which was filed on Apr. 23, 2004.

### BACKGROUND OF THE INVENTION

The vitamin replacement as a hangover ameliorative relates to over the counter medicines generally and more specifically to hangover remedies. A unique aspect of the present invention is consuming vitamins that alcohol later destroys.

People have brewed and consumed alcohol since before the Roman Empire. In moderation, alcohol has tolerable effects. In large quantities, alcohol causes a person to feel ill and to have headaches, commonly called a hangover. People have many folk remedies for hangovers such as aspirin, cold showers, and black coffee. However, a hangover has systemic effects only cured by time. In people, the liver metabolizes alcohol at a fixed rate. Folk remedies treat the symptoms of a hangover but only metabolization removes alcohol from a person. During alcohol metabolization, the liver consumes B vitamins and other nutrients. The decrease in nutrient levels makes a person feel the symptoms of a hangover. The present art overcomes the limitations of the prior art. That is, the art of the present invention, a vitamin replacement as a hangover ameliorative, buffers the drop in nutrient levels during metabolizing of alcohol.

The difficulty in providing a vitamin replacement as a hangover ameliorative is shown by a typical hangover remedy. On an occasion, a person imbibes an alcoholic beverage, sometimes to excess. Roused from sleep, a hung-over person seeks black coffee. The black coffee warms the person with its heat and stimulates the person with its caffeine. However, the person usually takes the coffee after getting hung-over and not as a preventive measure. The hung-over person endures depressed nutrient levels while his liver processes the alcohol.

### SUMMARY OF THE INVENTION

An object of the present invention is to provide vitamin pills that reduce the effects of alcohol consumption, commonly called a hangover, by nutrient preloading and replenishment.

Another object of the vitamin replacement as a hangover ameliorative is to provide pills readily consumed by drinkers of alcohol.

A still another object of the vitamin replacement as a hangover ameliorative is to metabolize alcohol more efficiently in the body.

A further object of the vitamin replacement as a hangover ameliorative is to maintain steady blood insulin levels in the body, reducing the effects of transient hypoglycemia.

A still further object of the vitamin replacement as a hangover ameliorative is to keep nerve transmissions steady in the body.

It is still a further object of the vitamin replacement as a hangover ameliorative that is easily and efficiently manufactured and marketed.

Lastly, it is an object of the present invention to provide a new and improved ameliorative that has a low cost of manufacture and which is then available at low prices to the consuming public.

These and other objects will become apparent to those skilled in the art in light of the following disclosure.

In accordance with the invention, generally stated, a vitamin series is provided that will increase nutrient levels and reduce the symptoms of a hangover. The vitamin series has four pills taken in two doses of two pills each. The pills comprise, by weight, about 270-330 mg thiamin (vitamin B1), about 216-264 mg riboflavin (vitamin B2), about 216-264 mg niacin (vitamin B3), about 216-264 mg panththenic acid (vitamin B5), about 216-264 mg pyridoxine (vitamin B6), about 0.6-1.4 mg (vitamin B7), about 216-264 mg pteroylmonoglutamic acid (vitamin B10), about 216-264 mg cyanocobalamine (vitamin B12), about 216-264 mg Inositol (vitamin B8 biotin), about 54-66 mg para-aminobenzoic acid (vitamin B11 PABA), about 216-264 mg choline, about 180-220 mg potassium, about 180-220 mg magnesium and about 630-770 mg conjugated linoleic acid. A person consumes two pills before a drinking session and two pills before sleeping following a drinking session.

### DETAILED DESCRIPTION

The present art overcomes the prior art limitations by stocking a person with nutrients consumed while metabolizing alcohol. The present invention consists of B vitamins, an omega-6-fatty acid, and electrolytic potassium and magnesium. The preferred embodiment has the ingredients at these weights:

| | | | |
|---|---|---|---|
| Vitamin B1 | 300 mg 8.9% | Inositol | 240 mg 7.1% |
| Vitamin B2 | 240 mg 7.1% | PABA | 60 mg 1.8% |
| Vitamin B3 | 240 mg 7.1% | Choline | 240 mg 7.1% |
| Vitamin B5 | 240 mg 7.1% | Potassium | 300 mg 8.9% |
| Vitamin B6 | 240 mg 7.1% | Magnesium | 300 mg 8.9% |
| Vitamin B10 | 240 mg 7.1% | Conj. Linoleic Acid | 500 mg 14.8% |
| Vitamin B12 | 240 mg 7.1% | | |

As components of the ameliorative, Thiamin (vitamin B1) assists in blood cell formation and metabolizing carbohydrates. Riboflavin (vitamin B2) helps the body to use oxygen and regulates the metabolism of amino acids, fatty acids, and carbohydrates. Riboflavin is further needed to activate pyridoxine (vitamin B6). Niacin (vitamin B3) assists in cell respiration and augments the release of energy from carbohydrates, fats, and proteins. Panththenic Acid (vitamin B5) has an important role in the secretion of hormones, such as cortisone, because of its role in supporting the adrenal gland. Pyridoxine (vitamin B6) balances the hormonal changes in women as well as assisting the immune system and the growth of new cells. Pyridoxine also assists in the processing and metabolism of proteins, fats and carbohydrates and with controlling mood and behavior. Pteroylmonoglutamic acid (vitamin B10) supports growth at many stages. Cyanocobalamine (vitamin B12) guides the manufacture and maintenance of red blood cells, stimulates appetite, promotes growth and releases energy.

Beyond the B vitamins, the ameliorative acquires its effect from other components. Inositol plays an important part in the health of cell membranes especially in the brain, bone marrow, eyes and intestines. PABA improves the use of protein in the body, forming of red blood cell, and the manufacture of folic acid in the intestines. Choline assists in weight control for people as well as cholesterol levels, keeping cell membranes healthy and in preventing gallstones. As a mineral, potassium helps in growth, building muscles, the transmission of nerve impulses, and control of heart activity. Magnesium aids in the formation of bone and teeth, and in the absorption of calcium and potassium. Where calcium stimulates the muscles, magnesium relaxes the muscles. Conjugated linoleic acid has recently been shown to dramatically reduce the deposition of fat within the body.

The pills are produced by combining the ingredients inside a shell. The shell is then closed and compressed. The ingredients attain a pill form suitable for oral consumption. The pills are packaged for resale to consumers.

To use the present invention, a person takes two pills with ten to twelve ounces of water before or just at the start of drinking alcohol. After consuming quantities of alcohol, the person takes two more pills with ten to twelve more ounces of water. The four pills together provide the dosage in the preferred embodiment. Alas, the present invention does not prevent hangovers completely. The present invention overloads a person with the nutrients destroyed by alcohol then, allows a person to drink without fear of the next day's hangover. The second dose adds more nutrients to a person to replace those nutrients lost during consumption and metabolization of alcohol. A person sleeps better following a drinking session. After sleeping, the person's nutrient deficiencies have greatly reduced, the person feels much better, and the person avoids a headache.

From the aforementioned description, a vitamin replacement as a hangover ameliorative has been described. The vitamin replacement as a hangover ameliorative is uniquely capable of providing nutrients to a person drinking and recovering from alcohol. The vitamin replacement as a hangover ameliorative and its various components may be manufactured from many materials including but not limited to B vitamins, nutritious minerals, binding agents, polymers, and composites.

1. A therapeutic composition comprising a mixture of ingredients including thiamin (vitamin B1), riboflavin (Vitamin B2), niacin (vitamin B3), panththenic Acid (vitamin B5), pyridoxine (vitamin B6), Vitamin B7, pteroylmonoglutamic acid (vitamin B10), cyanocobalamine (vitamin B12), Inositol, para-aminobenzoic acid, choline, potassium, magnesium, and conjugated linoleic acid.

2. The therapeutic composition of claim 1 and further comprising about 270-330 mg thiamin (vitamin B1), about 216-264 mg riboflavin (vitamin B2), about 216-264 mg niacin (vitamin B3), about 216-264 mg panththenic acid (vitamin B5), about 216-264 mg pyridoxine (vitamin B6), about 0.6-1.4 mg (vitamin B7), about 216-264 mg pteroylmonoglutamic acid (vitamin B10), about 216-264 mg cyanocobalamine (vitamin B12), about 216-264 mg Inositol (vitamin B8 biotin), about 54-66 mg para-aminobenzoic acid (vitamin B11 PABA), about 216-264 mg choline, about 180-220 mg potassium, about 180-220 mg magnesium and about 630-770 mg conjugated linoleic acid.

3. The therapeutic composition of claim 2 wherein the composition is a pill, and the pill is formed by ingredients compressed into a pill shape.
